(19) United States
(12) Patent Application Publication
(10) Pub. No.: US 2014/0275224 A1
(43) Pub. Date: Sep. 18, 2014

(54) CYTOSINE DEAMINASE MODULATORS FOR ENHANCEMENT OF DNA TRANSFECTION

(71) Applicants: Reuben S. Harris, Saint Paul, MN (US); Daniel A. Harki, Minneapolis, MN (US); Angela L. Perkins-Harki, Minneapolis, MN (US); Michael A. Carpenter, Minneapolis, MN (US); Ming Li, Saint Paul, MN (US)

(72) Inventors: Reuben S. Harris, Saint Paul, MN (US); Daniel A. Harki, Minneapolis, MN (US); Angela L. Perkins-Harki, Minneapolis, MN (US); Michael A. Carpenter, Minneapolis, MN (US); Ming Li, Saint Paul, MN (US)

(73) Assignee: Regents of the University of Minnesota, St. Paul, MN (US)

(21) Appl. No.: 13/883,267

(22) PCT Filed: Nov. 3, 2011
(86) PCT No.: PCT/US2011/059178

§ 371 Date: May 15, 2014

Related U.S. Application Data
(60) Provisional application No. 61/410,482, filed on Nov. 5, 2010.

(51) Int. Cl. C07D 417/14; A61K 31/4709; C07D 417/12; C07D 235/28; A61K 31/4184; A61K 31/4439; A61K 31/428; C07D 235/18; C07D 277/66; A61K 31/433; C07D 285/08; A61K 31/713; C07D 277/74

(52) U.S. Cl. CPC C07D 417/14; A61K 31/713; A61K 31/4709; C07D 417/12; C07D 235/28; A61K 31/4184; C07D 277/74; A61K 31/428; C07D 235/18; C07D 277/66; A61K 31/433; C07D 285/08; A61K 31/4439
USPC 514/44 R; 546/167; 548/307.1; 544/135; 548/310.7; 548/179; 548/129; 546/268.7; 435/184

(57) ABSTRACT

Compounds and methods are provided for enhancing or boosting the transfection rate or efficiency of mammalian cells by foreign DNA, such as bacterial plasmid DNA. Compounds, including natural products and inventive synthetic compounds can increase the effectiveness of uptake and incorporation of foreign DNA by mammalian cells, such as human cells, by suppression of DNA cytosine deamination, which is believed to be a mechanism by which these cells eliminate foreign DNA. Inhibition of the cytosine deaminase enzymes by compounds as described herein serves to provide more effective transfection of eukaryotic cells by plasmids including engineered gene sequences. Transfection can be used to study cellular processes, or to cure genetic diseases in human patients. The inventive materials and methods increase the efficiency and effectiveness of such transfection techniques.

### CROSS-REFERENCE TO RELATED APPLICATIONS

This application claims the priority of U.S. Ser. No. 61/410,482, filed on Nov. 5, 2010, which is incorporated herein by reference in its entirety.

### STATEMENT OF GOVERNMENT SUPPORT

This invention was made with government support under grant number R03MH089432, awarded by the National Institutes of Health. The U.S. government has certain rights in the invention.

### BACKGROUND

Human cells differ enormously in their ability to be transfected with foreign DNA such as plasmids. Transfection efficiencies, range from over 50% for human embryonic kidney (HEK)-293 cells with modern lipid-based reagents to lower than 1% of primary human monocytes with even the most refined gene transfer technologies (e.g., nucleofection). It is therefore no surprise that HEK-293 cells are the preferred workhorse of many laboratories because they readily enable many studies that rely on highly efficient transfection including protein (co)localization, protein purification, genetic complementation and virus production. In contrast, several common lymphoid cell types such as T cells, B cells and monocytes and many other primary cell types are much more difficult to transfect and therefore are rarely used in these studies even if they may provide more relevant model systems. One molecular reason for this difference in transfection efficiency lies in the expression levels of APOBEC3 (A3) proteins; HEK-293 cells express low A3 levels whereas most other cell lines and primary cell types express much higher A3 levels (23).

Human cells can express up to seven distinct A3 proteins A3A, A3B, A3C, A3D (also known as A3DE), A3F, A3G, and A3H (23). Related polynucleotide cytosine deaminases include AID (present in all vertebrates) and APOBEC1 (present in most vertebrates). All A3 proteins in mammals are comprised of either one or two conserved zinc-coordinating catalytic domains, each of which belongs to one of three distinct phylogenetic classes Z1, Z2, or Z3 (35, 36). Each of these proteins has elicited DNA cytosine to uracil (C-to-U) deaminase activity in a variety of assay systems [e.g., (1, 8, 6, 7, 28)]. The physiological functions of these proteins are still under intense investigation, but most studies point toward fundamental roles in innate immunity by blocking the spread of parasitic retroviruses, retrotransposons, and DNA-based viruses such as Hepatitis B Virus and Human Papilloma Virus [reviewed by (1, 8, 27, 29)]. It has been discovered that the modification and clearance of naked, foreign double-stranded DNA from human cells is mediated by these cytosine deaminase enzymes (28). Foreign DNA poses a threat to the integrity of every cell, and can be defined as any DNA that exists outside of the normal cellular blueprint. Examples of foreign DNA include truly foreign DNAs such as those from bacteria, fungi, or viruses, but they may also include DNAs from apoptotic or necrotic human cells (16).

One significant class of foreign DNA that can be useful for introduction into mammalian cells is plasmid DNA, which is used frequently for the genetic engineering of target cells through the process of transfection. Plasmid DNA prepared from the bacterium E. coli provides the means to accomplish a large fraction of all human biomedical research. We have demonstrated that most A3 proteins, and particularly A3A, trigger the clearance of transfected plasmid DNA from human cells (28). These data led us to propose a working model in which A3 proteins irreversibly ‘modify’ foreign DNA by deaminating cytosines to uracils (FIG. 1) (28). Subsequent excision of the DNA U's by the uracil DNA glycosylase UNG2 results in foreign DNA degradation and clearance (28). Taken together with the broad A3 expression profile in human tissues (23), we hypothesize that the A3 proteins provide a major barrier to foreign DNA transfer into all types of mammalian cells. This function has been likened to that of bacterial restriction endonucleases, which prevent interspecies DNA transmission and bacteriophage infection (3).

### SUMMARY

The present invention is directed to materials and methods, based on this discovery of a new physiological function for the family of DNA polynucleotide cytosine deaminases, that can be used to enhance the efficiency of transfection of eukaryotic cells, such as with plasmid DNA containing genetically engineered sequences for expression in the target cells. Target cells can be any eukaryotic cell, including but not limited to vertebrate cells such as, mammalian cells, primate cells, and human cells; or invertebrate cells, such as arthropod (e.g., Drosophila) or nematode (e.g., C. elegans) cells.

In various embodiments the present invention provides chemical compounds that can serve as enhancers or boosters for increasing the efficiency and/or fidelity of transfection or transduction of eukaryotic cells, such as mammalian cells, with foreign DNA. The compounds can act as modulators of one or more of the family of DNA polynucleotide cytosine deaminase enzymes (“cytosine deaminases”), for example, of vertebrate cytosine deaminases (e.g., APOBEC3 [A3]-family cytosine deaminases, such as APOBEC3A [A3A], APOBEC3B [A3B], APOBEC3C [A3C], APOBEC3D [A3D], APOBEC3F [A3F], APOBEC3G [A3G], and APOBEC3H [A3H] any Z1-, Z2-, and/or Z3-type A3, and related enzymes such as AID and APOBEC1). By use of the inventive compounds, clearance of foreign DNA by the cell is diminished, such that a greater fidelity and efficiency of uptake and integration of the foreign DNA into the cellular genome occurs. The foreign DNA can include single-stranded or double-stranded DNA fragments, plasmids, cosmids, synthetic chromosomes, engineered viral DNA, and the like. The uptake of the foreign DNA can include processes of transfection, such as of target cells by engineered bacterial plasmids and the like, and the process of transduction, such as of target cells by engineered viruses or virus-like entities. Compounds of the invention can be used to improve the fidelity and uptake of the foreign DNA by the target cell in conjunction with use of known materials and methods for carrying out the methods of transfection and transduction such as transfection adjuvants (e.g., cationic lipids, cationic polymers, cationic peptides, pegylated liposomes, etc.; see J. Nedderwald, Tackling Transfection's Complexity, Gen. Eng. Biotech. News, Sep. 1, 2010, pp. 46-48), and/or electroporation. Without wishing to be bound by theory, the compounds are believed to act on cytosine deaminase enzymes to accomplish this end. Compounds of the invention can act to reduce the degradation of foreign DNA by the target cell, it is believed through the modulation of a cytosine deaminase. By reducing the degradation of foreign DNA, the foreign DNA can more efficiently and with greater fidelity be expressed by the target cell, whether integrated into the target cell genome or persisting as extrachromosomal DNA.

In an embodiment, the invention provides a compound of formula (IA) or (IB)

[chemical structure]

wherein R is hydrogen, alkyl, cycloalkyl, heterocyclyl, aryl, or heteroaryl, wherein any alkyl, cycloalkyl, heterocyclyl, aryl, or heteroaryl is substituted with 0-4 J;

the ring comprising X¹-X⁴ is present or absent; when absent, the ring comprising Y¹ and Y² is further substituted with R; when present, each of X¹-X⁴ is an independently selected C or N, provided that when any of X¹-X⁴ is N, the respective R¹-R⁴ is absent;

each of R¹-R⁴, when present, is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

W is O or S;

Y¹ and Y² are independently N, O, S, or CR;

each of Z¹-Z³ is an independently selected CR⁵, CR⁵═CR⁵, N, or N═N;

each R⁵ is independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)N(R′)N(R′)C(O)R′, N(R′)N(R′)C(O)OR′, N(R′)N(R′)CON(R′)₂, N(R′)SO₂R′, N(R′)SO₂N(R′)₂, N(R)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof;

provided the compound of formula (IA) or (IB) is not

[chemical structure]

and provided that the compound of formula (IA) is not a compound that when Y¹ is N, Y² is CH, Z¹ is CR⁵, Z² and Z³ are both N, and R is phenyl, then R⁵ is hydrogen, halo, unsubstituted phenyl, unsubstituted furan-2-yl, unsubstituted pyridin-4-yl, or unsubstituted tetrahydrofuran-2-yl, or when R is hydrogen, then R⁵ is furan-3-yl, or pyridin-4-yl.

In an embodiment, the invention provides a compound of formula (II)

[chemical structure]

wherein each of X¹-X⁴ is an independently selected C or N, provided that when any of X¹-X⁴ is N, the respective R¹-R⁴ is absent; when present, each of R¹-R⁴ is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

W¹ is O, S, or CH₂;

W² is O or S;

Y¹ and Y² are independently selected O, S, or NR;

each R is independently selected hydrogen or alkyl;

Ar is aryl or heteroaryl, substituted with 0-4 J;

n is 1 to about 6;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof;

provided the compound of formula (II) is not

[chemical structure]

In an embodiment, the invention provides a compound of formula (III)

[chemical structure]

wherein each of X¹-X⁴ is an independently selected C or N, provided that when any of X¹-X⁴ is N, the respective R¹-R⁴ is absent; when present, each of R¹-R⁴ is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

Y is O or S;

R⁵ is alkyl, cycloalkyl, aryl, heterocyclyl or heteroaryl, substituted with 0-4 J;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)ₐ₂N(R′)N(R′)₂, N(R′)N(R′)C(O)R′, N(R′)N(R′)C(O)OR′, N(R′)N(R′)CON(R′)₂, N(R)SO₂R′, N(R′)SO₂N(R′)₂, N(R)C(O)OR′, N(R)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof;

provided the compound of formula (III) is not

[chemical structure]

In an embodiment, the invention provides a compound of the formula (IV)

[chemical structure]

wherein X¹ and X² are each independently C or N, provided that when X¹ or X² is N, the respective R¹ or R² is absent; when present, R¹ and R² each is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J; or R¹ and R² together with X¹ and X² form an optionally substituted cycloalkyl, heterocyclyl, aryl or heteroaryl ring;

W is O, S, or CH₂;

Y is O or S;

Z is absent or is NR;

each R is independently selected H or alkyl;

n1 and n2 are each independently 0 to 6;

Ar is aryl or heteroaryl, substituted with 0-4 J;

J is F, Cl, Br, I, OR′, OC(O)N(R)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R)₂, OC(O)N(R′)₂, C(S)N(R′)₂, (CH₂)₀₋₂N(R′)C(O)R′, (CH₂)₀₋₂N(R′)N(R′)₂, N(R′)N(R)C(O)R′, N(R′)N(R′)C(O)OR′, N(R′)N(R′)CON(R′)₂, N(R′)SO₂R′, N(R′)SO₂N(R′)₂, N(R)C(O)OR′, N(R)C(O)R′, N(R′)C(S)R′, N(R′)C(O)N(R′)₂, N(R)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof;

provided the compound of formula (IV) is not

[chemical structure]

In an embodiment, the invention provides a compound of formula (V)

[chemical structure]

wherein each independently selected R, R⁵, Y¹ and Y², are as defined for the compound of formula (I); or any salt thereof.

In an embodiment, the invention provides a compound of formula (VI)

[chemical structure]

wherein R⁵ is as defined for the compound of formula (I), and R⁶ is hydrogen, alkylcarbonyl, cycloalkylcarbonyl, aroyl, or heteroaroyl; or a salt thereof.

In an embodiment, the invention provides a compound of formula (VII)

[chemical structure]

wherein R is hydrogen, alkyl, cycloalkyl, heterocyclyl, aryl, or heteroaryl, wherein any alkyl, cycloalkyl, heterocyclyl, aryl, or heteroaryl is substituted with 0-4 J;

the ring comprising X¹-X⁴ is present or absent; when absent, the ring comprising Y¹ and Y² is further substituted with R; when present, each of X¹-X⁴ is an independently selected C or N, provided that when any of X¹-X⁴ is N, the respective R¹-R⁴ is absent;

each of R¹-R⁴, when present, is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

Y¹ and Y² independently N, O, S, or CR; and

R⁷ comprises OH, OR, or N(R)R⁸, wherein R⁸ is aminoalkyl, mono- or di-alkylaminoalkyl, heterocyclylalkyl, or heteroarylalkyl; or a salt thereof.

In various embodiments, the invention provides a method of inhibiting a DNA polynucleotide cytosine deaminase, which can be a vertebrate, invertebrate, mammalian, or human cytosine deaminase, comprising contacting the deaminase with an effective amount or concentration of any of:

(a) a compound of formula (IA) or (IB)

[chemical structure]

wherein R is hydrogen, alkyl, cycloalkyl, heterocyclyl, aryl, or heteroaryl, wherein any alkyl, cycloalkyl, heterocyclyl, aryl, or heteroaryl is substituted with 0-4 J; the ring comprising X¹-X⁴ is present or absent; when absent, the ring comprising Y¹ and Y² is further substituted with R; when present, each of X¹-X⁴ is an independently selected C or N, provided that when any of X¹-X⁴ is N, the respective R¹-R⁴ is absent;

each of R¹-R⁴, when present, is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

W is O or S;

Y¹ and Y² are independently N, O, S, or CR;

each of Z¹-Z³ is an independently selected CR⁵, CR⁵═CR⁵, N, or N═N;

each R⁵ is independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof; or,

(b) a compound of formula (II)

[chemical structure]

wherein each of X¹-X⁴ is an independently selected C or N, provided that when any of X¹-X⁴ is N, the respective R¹-R⁴ is absent; when present, each of R¹-R⁴ is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

W¹ is O, S, or CH₂;

W² is O or S;

Y¹ and Y² are independently selected O, S, or NR;

each R is independently selected hydrogen or alkyl;

Ar is aryl or heteroaryl, substituted with 0-4 J;

n is 1 to about 6;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof; or,

(c) a compound of formula (III)

[chemical structure]

wherein each of X¹-X⁴ is an independently selected C or N, provided that when any of X¹-X⁴ is N, the respective R¹-R⁴ is absent; when present, each of R¹-R⁴ is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

Y is O or S;

R⁵ is alkyl, cycloalkyl, aryl, heterocyclyl or heteroaryl, substituted with 0-4 J;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof; or,

(d) a compound of the formula (IV)

[chemical structure]

wherein X¹ and X² are each independently C or N, provided that when X¹ or X² is N, the respective R¹ or R² is absent; when present, R¹ and R² each is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J; or R¹ and R² together with X¹ and X² form an optionally substituted cycloalkyl, heterocyclyl, aryl or heteroaryl ring;

W is O, S, or CH₂;

Y is O or S;

Z is absent or is NR;

each R is independently selected H or alkyl;

n1 and n2 are each independently 0 to 6;

Ar is aryl or heteroaryl, substituted with 0-4 J;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof; or,

(e) a compound of formula (V)

[chemical structure]

wherein each independently selected R, R⁵, Y¹ and Y², are as defined for the compound of formula (I); or any salt thereof; or,

(f) a compound of formula (VI)

[chemical structure]

wherein R⁵ is as defined for the compound of formula (I), and R⁶ is hydrogen, alkylcarbonyl, cycloalkylcarbonyl, aroyl, or heteroaroyl; or a salt thereof; or,

(g) a compound of formula (VII)

[chemical structure]

wherein R, X¹-X⁴, R¹-R⁴, Y¹, and Y², are as defined as for the compound of formula (I), and R² comprises OH, OR, or N(R)R⁶, wherein R⁸ is aminoalkyl, mono- or di-alkylaminoalkyl, heterocyclylalkyl, or heteroarylalkyl; or a salt thereof; or,

(h) any of the following compounds:

[chemical structure]

[chemical structure]

[chemical structure]

[chemical structure]

[chemical structure]

[chemical structure]

[chemical structure]

[chemical structure]

[chemical structure]

or any salt thereof. The cytosine deaminase can be a vertebrate cytosine deaminase; more specifically the cytosine deaminase can be a mammalian DNA polynucleotide cytosine deaminase, for example the cytosine deaminase can be any APOBEC3-family enzyme, such as A3A, A3B, A3C, A3D, A3E, A3F, A3G, any of Z1-, Z2-, and/or Z3-type protein, or AID or APOBEC1.

In various embodiments, the invention provides a method of inhibiting the degradation of foreign DNA within a eukaryotic cell, comprising contacting the cell comprising the foreign DNA, under conditions suitable for transfection or transduction to occur, with an effective amount or concentration of a compound of the invention. The eukaryotic cell can be a vertebrate cell such as a mammalian cell, a primate cell, or a human cell; or can be an invertebrate cell, such as an arthropod (e.g., Drosophila) or a nematode (e.g., C. elegans) cell. The foreign DNA can include single-stranded or double-stranded DNA fragments, plasmids, cosmids, synthetic chromosomes, engineered viral DNA, and the like. The contacting can be carried out using materials and methods known in the art as transfection or transduction adjuvants, such as cationic lipids, cationic polymers, cationic peptides, pegylated liposomes, electroporation, and the like.

In various embodiments, the invention provides methods of increasing or enhancing the efficiency or fidelity of DNA transfection of mammalian cells by foreign DNA, such as plasmid DNA, etc., which can include genetically engineered sequences of various types, the method comprising contacting the mammalian cell, or any other kind of eukaryotic cell, with the foreign DNA, under conditions suitable for transfection or transduction to occur, such as the plasmid DNA, in the presence of an effective cytosine deaminase inhibitory amount of any of the above-listed cytosine deaminase inhibitory compounds. In various embodiments, the invention provides a method to improve the transfection efficiency of mammalian cells, either in vivo or in vitro, wherein the cells are engineered with a greater efficiency and fidelity, i.e., a higher transfection rate, compared to art methods. The foreign DNA can include single-stranded or double-stranded DNA fragments, plasmids, cosmids, synthetic chromosomes, engineered viral DNA, and the like. The contacting can be carried out using materials and methods known in the art as transfection or transduction adjuvants, such as cationic lipids, cationic polymers, cationic peptides, pegylated liposomes, electroporation, and the like.

In various embodiments, the invention provides a method of treating a genetic disease in a patient afflicted therewith, the method comprising contacting a cell or tissue in vivo in the body of a patient afflicted with the genetic disease with a curative DNA, under conditions suitable for transfection or transduction to occur, in the presence of an effective cytosine deaminase inhibitory amount of any of the above-listed cytosine deaminase inhibitory compounds, using methods and materials as are known in the art.

In various embodiments, the invention provides a kit comprising a compound of the invention and instructional material, further optionally comprising foreign DNA and a transfection adjuvant, for transfection of a target cell.

### BRIEF DESCRIPTION OF THE FIGURES

FIG. 1 illustrates a working model where A3 proteins irreversibly ‘modify’ foreign DNA by deaminating cytosines to uracils;

FIG. 2A illustrates a schematic of fluorescence-based DNA deamination assay;

FIG. 2B illustrates dose responses for the compounds of the invention against A3A and A3G;

FIG. 2C illustrates dose responses for the compounds of the invention against UDG;

FIG. 3A is a schematic of the GFP-based transient transfection assay for foreign DNA restriction;

FIG. 3B illustrates restriction ratios for 50 μM of the compounds of the invention;

FIG. 4A represents Coomassie blue stained SDS-PAGE gel of purified A3G;

FIG. 4B represents Coomassie blue stained SDS-PAGE gel of purified A3A;

FIG. 4C illustrates deamination activity assay of purified A3G;

FIG. 4D illustrates deamination activity assay of purified A3A;

FIG. 5 illustrates inhibitory responses in A3A-expressing cell lysates of exemplary compounds;

FIG. 6A illustrates a rapid decline in transient GFP expression caused by A3A (Stenglein et al. (28));

FIG. 6B illustrates ability of A3A inhibitors of the invention enhancing the transfection efficiency;

FIG. 7A is a schematic of the experimental procedure;

FIG. 7B illustrates representative data for primary human monocytes nucleofected with a GFP+ plasmid and then treated with DMSO or MN132 at the indicated concentrations;

FIG. 8A is a schematic of the experimental procedure illustrating inhibition of foreign DNA restriction;

FIG. 8B illustrates restriction ratio of an exemplary compound of the invention;

FIG. 8C illustrates restriction ratio of another exemplary compound of the invention; and

FIG. 8D illustrates restriction ratio of yet another exemplary compound of the invention.

### DETAILED DESCRIPTION

### Definitions

As used in the specification and the appended claims, the singular forms “a,” “an” and “the” include plural referents unless the context clearly dictates otherwise.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

As used herein, “individual” (as in the subject of the treatment) means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The term “disease” or “disorder” or “malcondition” are used interchangeably, and are used to refer to diseases or conditions wherein a genetic defect plays a role in the biochemical mechanisms involved in the disease or malcondition such that a therapeutically beneficial effect can be achieved by acting on the genetic defect, such as by gene therapy involving transformation one or more cells with foreign DNA that cures the genetic defect.

The expression “effective amount”, refers to the amount of a compound of the invention that is effective to enhance or improve the uptake and integration of foreign DNA, such as bacterial plasmid DNA, in the process of transfection a target or host cell, such as a mammalian cell targeted for genetic engineering.

By “transfection” is meant the uptake of foreign DNA by a eukaryotic cell, such as the uptake of bacterial plasmid DNA by a mammalian cell. The plasmid DNA can contain engineered DNA sequences, which can then be taken up, integrated, and expressed in the target mammalian cell (ie. stable transfection). Transfected plasmids may also be expressed transiently, without integration (transient transfection).

By “transduction” is meant the uptake of foreign DNA by a eukaryotic cell through a virally mediated process, wherein the viral mechanisms for transporting nucleic acids through cell and nuclear membranes are used to introduce engineered DNA packaged within a viral particle.

When a compound of the invention is said to be “transfection enhancing”, it is intended that the compound can also be understood to be “transduction enhancing”, in that both techniques involve incorporation of foreign DNA into target cells, but using different vectors. In both situations, it is believed that the compounds of the invention act to inhibit the degradation of foreign DNA within a target cell through inhibition of cytosine deaminases that would otherwise break down the foreign DNA. A compound is transfection enhancing if it increases the rate, efficiency, or fidelity of uptake of the foreign DNA into the target cell relative to the rate, efficiency, or fidelity of uptake of the foreign DNA in the absence of the compound.

“Foreign DNA” as the term is used herein refers to any DNA that is not from the target cell but is introduced into the target cell by transfection or transduction. Such DNA can include single-stranded and double-stranded DNA fragments, plasmids, cosmids, synthetic chromosomes, and the like.

A “modulator” is a compound that acts on an enzyme or receptor to alter the bioactivity of the enzyme or receptor, directly or indirectly. For example, a modulator can be an inhibitor, agonist, antagonist, allosteric regulator, or the like.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

When a substituent is specified to be an atom or atoms of specified identity, “or a bond”, a configuration is referred to when the substituent is “a bond” that the groups that are immediately adjacent to the specified substituent are directly connected to each other in a chemically feasible bonding-configuration.

All chiral, diastereomeric, racemic forms of a structure are intended, unless a particular stereochemistry or isomeric form is specifically indicated. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

In general, “substituted” refers to an organic group as defined herein in which one or more bonds to a hydrogen atom contained therein are replaced by one or more bonds to a non-hydrogen atom such as, but not limited to, a halogen (i.e., F, CI, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted.

When a substituent is monovalent, such as, for example, F or Cl, it is bonded to the atom it is substituting by a single bond. When a substituent is more than monovalent, such as 0, which is divalent, it can be bonded to the atom it is substituting by more than one bond, i.e., a divalent substituent is bonded by a double bond; for example, a C substituted with 0 forms a carbonyl group, C═O, which can also be written as “CO”, “C(O)”, or “C(═O)”, wherein the C and the 0 are double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” group.

Alternatively, a divalent substituent such as O, S, C(O), S(O), or S(O)₂ can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)ₙ wherein n is 1, 2, 3, or more, and each R′ is independently selected.

C(O) and S(O)₂ groups can be bound to one or two heteroatoms, such as nitrogen, rather than to a carbon atom. For example, when a C(O) group is bound to one carbon and one nitrogen atom, the resulting group is called an “amide” or “carboxamide.” When a C(O) group is bound to two nitrogen atoms, the functional group is termed a urea. When a S(O)₂ group is bound to one carbon and one nitrogen atom, the resulting unit is termed a “sulfonamide.” When a S(O)₂ group is bound to two nitrogen atoms, the resulting unit is termed a “sulfamate.”

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

As to any of the groups described herein, which contain one or more substituents, it is understood, of course, that such groups do not contain any substitution or substitution patterns which are sterically impractical and/or synthetically nonfeasible. In addition, the compounds of this disclosed subject matter include all stereochemical isomers arising from the substitution of these compounds.

Selected substituents within the compounds described herein are present to a recursive degree. In this context, “recursive substituent” means that a substituent may recite another instance of itself or of another substituent that itself recites the first substituent. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the disclosed subject matter. One of ordinary skill in the art of medicinal and organic chemistry understands the versatility of such substituents. To the degree that recursive substituents are present in a claim of the disclosed subject matter, the total number should be determined as set forth above.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic,” “carbocyclyl,” and “carbocycle” denote a ring structure wherein the atoms of the ring are carbon, such as a cycloalkyl group or an aryl group. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N-1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above. A carbocyclyl ring can be a cycloalkyl ring, a cycloalkenyl ring, or an aryl ring. A carbocyclyl can be monocyclic or polycyclic, and if polycyclic each ring can be independently be a cycloalkyl ring, a cycloalkenyl ring, or an aryl ring.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂CH₂—OH, —CH₂—CH₂—NH—CH₃, —CH₂—S—CH₂—CH₃, —CH₂CH₂—S(═O)—CH₃, and —CH₂CH₂—O—CH₂CH₂—O—CH₃. Up to two heteroatoms may be consecutive, such as, for example, —CH₂—NH—OCH₃, or CH₂—CH₂—S—S—CH₃.

A “cycloheteroalkyl” ring is a cycloalkyl ring containing at least one heteroatom. A cycloheteroalkyl ring can also be termed a “heterocyclyl,” described below.

The term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, —CH₂—CH═CH—CH₂—SH, and —CH═CH—O—CH₂CH₂—O—CH₃.

Aryl groups are cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups or the term “heterocyclyl” includes aromatic and non-aromatic ring compounds containing 3 or more ring members, of which, one or more is a heteroatom such as, but not limited to, N, O, and S. Thus a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclylcan be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl-1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[bf]azepine-5-yl), and the like.

Heterocyclylalkyl groups are alkyl groups as defined above in which a hydrogen or carbon bond of an alkyl group as defined above is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structures are substituted therewith.

The terms “halo” or “halogen” or “halide” by themselves or as part of another substituent mean, unless otherwise stated, a fluorine, chlorine, bromine, or iodine atom, preferably, fluorine, chlorine, or bromine.

A “haloalkyl” group includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

A “haloalkoxy” group includes mono-halo alkoxy groups, poly-halo alkoxy groups wherein all halo atoms can be the same or different, and per-halo alkoxy groups, wherein all hydrogen atoms are replaced by halogen atoms, such as fluoro. Examples of haloalkoxy include trifluoromethoxy, 1,1-dichloroethoxy, 1,2-dichloroethoxy, 1,3-dibromo-3,3-difluoropropoxy, perfluorobutoxy, and the like.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is CF₃.

The term “(Cₓ-C_{y})perfluoroalkylene,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkylene, more preferred is —(C₁-C₃)perfluoroalkylene, most preferred is CF₂.

The terms “aryloxy” and “arylalkoxy” refer to, respectively, an aryl group bonded to an oxygen atom and an aralkyl group bonded to the oxygen atom at the alkyl moiety. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” includes primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

An “amino” group is a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary or quaternary amino group. An “alkylamino” group includes a monoalkylamino, dialkylamino, and trialkylamino group.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)NR₂, and NRC(O)R groups, respectively. Amide groups therefore include but are not limited to primary carboxamide groups (—C(O)NH₂) and formamide groups (—NHC(O)H). A “carboxamido” group is a group of the formula C(O)NR₂, wherein R can be H, alkyl, aryl, etc.

The term “azido” refers to an N₃ group. An “azide” can be an organic azide or can be a salt of the azide (N₃⁻) anion. The term “nitro” refers to an NO₂ group bonded to an organic moiety. The term “nitroso” refers to an NO group bonded to an organic moiety. The term nitrate refers to an ONO₂ group bonded to an organic moiety or to a salt of the nitrate (NO₃⁻) anion.

The term “urethane” (“carbamoyl” or “carbamyl”) includes N- and O-urethane groups, i.e., —NRC(O)OR and OC(O)NR₂ groups, respectively.

The term “sulfonamide” (or “sulfonamido”) includes S- and N-sulfonamide groups, i.e., —SO₂NR₂ and NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is C(NH)NH₂.

The term “guanidine” or “guanidino” includes groups of the formula —NRC(NR)NR₂. Typically, a guanidino group is NHC(NH)NH₂.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium: potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

A “hydrate” is a compound that exists in a composition with water molecules. The composition can include water in stoichiometic quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometic or non-stoichiometric. As the term is used herein a “solvate” refers to a solid form, i.e., a compound in solution in a solvent, while it may be solvated, is not a solvate as the term is used herein.

A “prodrug” as is well known in the art is a substance that can be administered to a patient where the substance is converted in vivo by the action of biochemicals within the patients body, such as enzymes, to the active pharmaceutical ingredient. Examples of prodrugs include esters of carboxylic acid groups, which can be hydrolyzed by endogenous esterases as are found in the bloodstream of humans and other mammals. Conventional procedures for the selection and preparation of suitable prodrug derivatives are described, for example, in “Design of Prodrugs”, ed. H. Bundgaard, Elsevier, 1985.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided.

Provisos may apply to any of the disclosed categories or embodiments wherein any one or more of the other above disclosed embodiments or species may be excluded from such categories or embodiments.

The present invention further embraces isolated compounds according to formula (I). The expression “isolated compound” refers to a preparation of a compound of formula (I), or a mixture of compounds according to formula (I), wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of formula (I) or a mixture of compounds according to formula (I), which contains the named compound or mixture of compounds according to formula (I) in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds of the invention and intermediates may be isolated from their reaction mixtures and purified by standard techniques such as filtration, liquid-liquid extraction, solid phase extraction, distillation, recrystallization or chromatography, including flash column chromatography, or HPLC.

### Isomerism and Tautomerism in Compounds of the Invention

### Tautomerism

Within the present invention it is to be understood that a compound of the formula (I) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

[chemical structure]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

[chemical structure]

is an example of tautomerism. Accordingly, a structure depicted herein as one tautomer is intended to also include the other tautomer.

### Optical Isomerism

It will be understood that when compounds of the present invention contain one or more chiral centers, the compounds may exist in, and may be isolated as pure enantiomeric or diastereomeric forms, or as racemic mixtures. The present invention therefore includes any possible enantiomers, diastereomers, racemates or mixtures thereof of the compounds of the invention.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

[chemical structure]

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound of the invention, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

In various embodiments, the compound or set of compounds, such as are among the inventive compounds or are used in the inventive methods, can be any one of any of the combinations and/or sub-combinations of the above-listed embodiments.

### DETAILED DESCRIPTION

The invention is directed to compounds and methods effective for enhancing the effectiveness of transfection or transduction of eukaryotic target cells by foreign DNA, such as by the DNA of bacterial plasmids. Target cells can be vertebrate cells, such as mammalian and human cells, or can be invertebrate cells, such as cells of insects (e.g., Drosophila) or nematodes (e.g., C. elegans). Some kinds of foreign DNA, such as plasmids, are well known to be useful as vectors for introducing engineered DNA sequences coding for desired genes and/or regulatory elements into target cells, which then express the engineered DNA. Compounds of the invention can act to improve the efficiency or rate of uptake and transformation of foreign DNA by target cells, and the fidelity of the incorporated DNA with respect to the introduced DNA. Also, in various embodiments, known compounds such as natural products can be used for this previously unknown function of enhancing transfection of target eukaryotic cells by foreign DNA. Contacting eukaryotic cells, such as mammalian or human cells, with an effective amount of the foreign DNA, such as an engineered plasmid, in the presence of an effective amount or concentration of a transfection enhancing compound as disclosed and claimed herein, can provide genetically transformed eukaryotic cells capable of expressing the introduced DNA. Such introduced DNA can be engineered either for purposes of investigating cell function, or for the purposes of genetic therapy, i.e., curing genetic diseases in patients by transforming some of the patient's cells with therapeutic foreign DNA. For example, genetic defects in a patient resulting from the absence or lack of expression of a gene that provides the body with a necessary component or enzyme can be cured by transfecting cells of the patient with curative DNA. Carrying out this transfection using compounds as disclosed and claimed herein for this purpose can improve the rate or efficiency of the transfection. The transfection or transduction can be carried out using materials and methods known in the art, but in the presence of effective amounts or concentrations of compounds of the invention or of compounds identified by the inventors herein as being useful for the purpose. Without wishing to be bound by theory, it is believed by the inventors herein that such enhancement of transfection occurs through the inhibition of cytosine deaminase enzymes, which serves to decrease the degradation of the foreign DNA within the target cell, leaving more of the foreign DNA available for expression, integration, etc.

In various embodiments, the invention provides a compound of formula (IA) or (IB)

[chemical structure]

wherein R is hydrogen, alkyl, cycloalkyl, heterocyclyl, aryl, or heteroaryl, wherein any alkyl, cycloalkyl, heterocyclyl, aryl, or heteroaryl is substituted with 0-4 J;

the ring comprising X¹-X⁴ is present or absent; when absent, the ring comprising Y¹ and Y² is further substituted with R; when present, each of X¹-X⁴ is an independently selected C or N, provided that when any of X¹-X⁴ is N, the respective R¹-R⁴ is absent;

each of R¹-R⁴, when present, is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

W is O or S;

Y¹ and Y² are independently N, O, S, or CR;

each of Z¹-Z³ is an independently selected CR⁵, CR⁵═CR⁵, N, or N═N;

each R⁵ is independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof;

provided the compound of formula (IA) or (IB) is not

[chemical structure]

and provided that the compound of formula (IA) is not a compound that when Y¹ is N, Y² is CH, Z¹ is CR⁵, Z² and Z³ are both N, and R is phenyl, then R⁵ is hydrogen, halo, unsubstituted phenyl, unsubstituted furan-2-yl, unsubstituted pyridin-4-yl, or unsubstituted tetrahydrofuran-2-yl, or when R is hydrogen, then R⁵ is furan-3-yl, or pyridin-4-yl.

In various embodiments, the compound of formula (IA) can comprise any of the following substituent variations:

[chemical structure]

wherein R and R⁵ are as defined herein, or can comprise any of the compounds of formula (IB) of analogous substitution pattern bearing an isomeric thiazole or the like.

In various embodiments, a compound of formula (IA) or (IB) can be any of the compounds shown in Table 1, below:

TABLE 1
Specifically Claimed Compounds of Formula (IA) and (IB)
C = comparative compound

| Cpd # | Structure | Formula |
|---|---|---|
| I-1 MN132 C | [chemical structure] | IA X¹-X⁴ = CH R = 5-methylfuran-2-yl Y¹ = N Y² = CH Z¹ = C Z² = N Z³ = N R⁵ = pyridi-4-yl |
| I-2 | [chemical structure] | IA X¹-X⁴ = CH R = phenyl Y¹ = N Y² = CH Z¹ = C Z² = N Z³ = N R⁵ = p-Cl-phenoxy- methyl |
| I-3 | [chemical structure] | IA X¹-X⁴ = CH R = 5-methylfuran-2-yl Y¹ = N Y² = CH Z¹ = C Z² = N Z³ = N R⁵ = ethyl |

Compounds of formula (IA) or (IB) can be prepared according to Scheme I, below, in conjunction with the knowledge of the person of ordinary skill in organic synthesis, including literature art, substituting appropriate reagents as needed to provide compounds with various substituent groups.

[chemical structure]

In various embodiments, the invention provides a compound of formula (II)

[chemical structure]

wherein each of X¹-X⁴ is an independently selected C or N, provided that when any of X¹-X⁴ is N, the respective R¹-R⁴ is absent; when present, each of R¹-R⁴ is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

W¹ is O, S, or CH₂;

W² is O or S;

Y¹ and Y² are independently selected O, S, or NR;

each R is independently selected hydrogen or alkyl;

Ar is aryl or heteroaryl, substituted with 0-4 J;

n is 1 to about 6;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R)N(R′)₂, N(R′)N(R′)C(O)R′, N(R)N(R)C(O)012′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof;

provided the compound of formula (II) is not

[chemical structure]

In various embodiments, the compound of formula (II) includes structural variants including those that can be introduced into the molecule according to Scheme IIa, below:

[chemical structure]

For example, compounds of formula 17 of Scheme IIa as shown can be couple with alkylxanthates such as 18 to provide thiobenzimidazole 14. Use of appropriately substituted phenylenediamines 17, or heteroaryl analogs thereof, can be incorporated into compounds of formula (II) using methods within ordinary skill. Further, the spacer region, an example of which is shown as compound 15, bromoacetic acid, can be prepared using analogous compounds with a carboxylic acid or protected carboxylate at one end, and a leaving group at the other end, such as are well known in the art. Similarly, the arylamino compound 16 can be varied, such as by using analogs with various substitution patterns, or heteroaryl analogs such as aminopyridines and the like, to provide structural variants falling within the definition of formula (II) herein.

Thiobenzimidazole 14 can be condensed with haloacetic acid 15 to yield a compound of formula

[chemical structure]

which can then be used in an amide-forming condensation with a compound 16 to provide a compound of formula (II). Alternatively, an intermediate of formula

[chemical structure]

can be prepared by condensing aniline 16 with an activated haloacetic acid 15. The halomethyl amide can then be reacted with thiobenzimidazole 14 to provide a compound of formula (II).

TABLE 2
Specifically Claimed Compounds of Formula (III)
C = comparative compound

| Cpd # | Structure | Formula |
|---|---|---|
| II-1 MN141 C | [chemical structure] | Y¹ = N Y² = N |
| II-2 | [chemical structure] | Y¹ = N Y² = N |
| II-3 | [chemical structure] | Y¹ = N Y² = N |
| II-4 | [chemical structure] | Y¹ = S Y² = N |

Compounds of formula (II) can be prepared according to Scheme II, below, in conjunction with the knowledge of the person of ordinary skill in organic synthesis, including literature art, substituting appropriate reagents as needed to provide compounds with various substituent groups.

[chemical structure]

In various embodiments, the invention provides a compound of formula (III)

[chemical structure]

wherein each of X¹-X⁴ is an independently selected C or N, provided that when any of X¹-X⁴ is N, the respective R¹-R⁴ is absent; when present, each of R¹-R⁴ is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

Y is O or S;

R⁵ is alkyl, cycloalkyl, aryl, heterocyclyl or heteroaryl, substituted with 0-4 J;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R)₂, OC(O)N(R)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof;

provided the compound of formula (III) is not

[chemical structure]

In various embodiments, the invention provides structural variants within formula (III) including:

[chemical structure]

Compounds of formula (III) can be prepared using ordinary skill in conjunction with the method of Scheme III:

[chemical structure]

Use of analogous intermediates can be employed to synthesize various embodiments of compounds of formula (III), as is apparent to the person of ordinary skill in the art. In an alternative approach, a compound of formula

[chemical structure]

can undergo a dehydrative ring closing reaction, such as with P₂O₅ or the like, to provide a compound of formula (III) wherein group Y is NH or S, respectively.

TABLE 3
Specifically Claimed Compounds of Formula (III)

| Cpd # | Structure | Formula |
|---|---|---|
| III-1 HARK 10005 | [chemical structure] | Y = N |
| III-2 MN152 C | [chemical structure] | Y = S |
| III-3 | [chemical structure] | Y = N |
| III-4 | [chemical structure] | Y = N |
| III-5 | [chemical structure] | Y = S |
| III-6 | [chemical structure] | Y = S |
| III-7 HARK 10028 | [chemical structure] | Y = N |
| III-8 | [chemical structure] | Y = S |

C = comparative compound

In various embodiments, the invention provides a compound of the formula (IV)

[chemical structure]

wherein X¹ and X² are each independently C or N, provided that when X¹ or X² is N, the respective R¹ or R² is absent; when present, R¹ and R² each is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J; or R¹ and R² together with X¹ and X² form an optionally substituted cycloalkyl, heterocyclyl, aryl or heteroaryl ring;

W is O, S, or CH₂;

Y is O or S;

Z is absent or is NR;

each R is independently selected H or alkyl;

n1 and n2 are each independently 0 to 6;

Ar is aryl or heteroaryl, substituted with 0-4 J;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R)N(R′)₂, N(R)N(R)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R)C(O)R′, N(R′)C(S)R′, N(R′)C(O)N(R′)₂, N(R)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof;

provided the compound of formula (IV) is not

[chemical structure]

In various embodiments, structural variants within formula (IV) can be prepared based on ordinary knowledge and according to the following Scheme IVa:

[chemical structure]

In various embodiments, analogs of compound 21 of Scheme IVa can be used to prepare aryl and heteroaryl substituted thiadiazoles such as the compound of formula 19. As above, analogs of compound 15 can be used for the spacer region, and various amino compounds, for example heteroarylalkylamines such as 20, can be used to assemble examples of compounds of formula (IV). For example, intermediate compounds such as

[chemical structure]

can be used in coupling reaction with the condensation product of intermediates 15 and 20, above in Scheme IV, such as a compound like

[chemical structure]

to provide compounds of formula (IV).

TABLE 4
Specifically Claimed Compounds of Formula (IV)

| Cpd # | Structure | Formula |
|---|---|---|
| IV-1 MN160 C | [chemical structure] | X¹ = N X² = C Y = S W = S Z = NH |
| IV-2 | [chemical structure] | X¹ = N X² = C Y = S W = S Z = NH |
| IV-3 | [chemical structure] | X¹ = N X² = C Y = S W = S Z = NH |
| IV-4 | [chemical structure] | X¹ = N X² = C Y = NH W = S Z = NH |
| IV-5 | [chemical structure] | X¹ = N X² = C Y = S W = S Z = NH |
| IV-6 | [chemical structure] | X¹ = N X² = C Y = NH W = S Z = NH |
| IV-7 | [chemical structure] | X¹ = N X² = C Y = S W = S Z = NH |

C = comparative compound

Compounds of formula (IV) can be prepared according to Scheme IV, below, in conjunction with the knowledge of the person of ordinary skill in organic synthesis, including literature art, substituting appropriate reagents as needed to provide compounds with various substituent groups.

[chemical structure]

In various embodiments, the invention provides compounds of formulas (V), (VI), and (VII).

For example, the invention provides a compound of formula (V)

[chemical structure]

wherein each independently selected R, R⁵, Y¹ and Y², are as defined for the compound of formula (I); or any salt thereof.

TABLE 5
Specifically Claimed Compounds of Formula (V)

| Cpd # | | |
|---|---|---|
| V-1 | [chemical structure] | Y¹ = N Y² = CH R⁵ = p-Cl-phenoxymethyl |

In various embodiments, the invention provides a compound of formula (VI)

[chemical structure]

wherein R⁵ is as defined for the compound of formula (I), and R⁶ is hydrogen, alkylcarbonyl, cycloalkylcarbonyl, aroyl, or heteroaroyl; or a salt thereof.

TABLE 6
Specifically Claimed Compounds of Formula (VI)

| Cpd # | Structure | Formula |
|---|---|---|
| VI-1 | [chemical structure] | R⁵ = p-Cl-phenoxymethyl R⁶ = acetyl |
| VI-2 | [chemical structure] | R⁵ = p-Cl-phenoxymethyl R⁶ = H |

Compounds of formula (V) or (VI) can be prepared according to Scheme V-VI, below, in conjunction with the knowledge of the person of ordinary skill in organic synthesis, including literature art, substituting appropriate reagents as needed to provide compounds with various substituent groups.

[chemical structure]

In various embodiments, the invention provides a compound of formula (VII)

[chemical structure]

wherein R, X¹-X⁴, R¹-R⁴, Y¹, and Y², are as defined as for the compound of formula (I), and R⁷ comprises OH, OR, or N(R)R⁸, wherein R⁸ is aminoalkyl, mono- or di-alkylaminoalkyl, heterocyclylalkyl, or heteroarylalkyl; or a salt thereof.

TABLE 7
Specifically Claimed Compounds of Formula (VII)

| Cpd # | Structure | Formula |
|---|---|---|
| VII-1 | [chemical structure] | X¹-X⁴ = CH Y¹ = N Y² = CH R = phenyl R⁷ = OH |
| VII-2 | [chemical structure] | X¹-X⁴ = CH Y¹ = N Y² = CH R = 5-methylfuran-2-yl R⁷ = OH |
| VII-3 | [chemical structure] | X¹-X⁴ = CH Y¹ = N Y² = CH R = 5-methylfuran-2-yl R⁷ = N,N-dimethylamino- ethylamino |
| VII-4 | [chemical structure] | X¹-X⁴ = CH Y¹ = N Y² = CH R = phenyl R⁷ = N,N-dimethylamino- ethylamino |
| VII-5 | [chemical structure] | X¹-X⁴ = CH Y¹ = N Y² = CH R = phenyl R⁷ = phenylamino |
| VII-6 | [chemical structure] | X¹-X⁴ = CH Y¹ = N Y² = CH R = phenyl R⁷ = N-methylbenzylamino |
| VII-7 | [chemical structure] | X¹-X⁴ = CH Y¹ = N Y² = CH R = phenyl R⁷ = 3-ethylphenylamino |
| VII-8 | [chemical structure] | X¹-X⁴ = CH Y¹ = N Y² = CH R = phenyl R⁷ = 3,5-dimethyl-phenylamino |
| VII-9 | [chemical structure] | X¹-X⁴ = CH Y¹ = N Y² = CH R = phenyl R⁷ = cycloheptylamino |
| VII-10 | [chemical structure] | X¹-X⁴ = CH Y¹ = N Y² = CH R = 5-methylfuran-2-yl R⁷ = 4-methylcarbonyl- piperidin-1-yl |
| VII-11 | [chemical structure] | X¹-X⁴ = CH Y¹ = N Y² = CH R = phenyl R⁷ = p-ethoxyphenylamino |
| VII-12 | [chemical structure] | X¹-X⁴ = CH Y¹ = N Y² = CH R = 5-methylfuran-2-yl R⁷ = p-ethoxyphenylamino |
| VII-13 | [chemical structure] | X¹-X⁴ = CH Y¹ = N Y² = CH R = furan-2-yl R⁷ = 4-methylcarbonyl-piperidin- 1-yl |
| VII-14 | [chemical structure] | X¹-X⁴ = CH Y¹ = N Y² = CH R = furan-2-yl R⁷ = 3-ethylcarbonyl-piperidin-1- yl |

Compounds of formulas (VII) can be synthesized according to the knowledge of the person of ordinary skill in the art according to Scheme VII.

[chemical structure]

In various embodiments, the invention provides a method of inhibiting a cytosine deaminase, comprising contacting the deaminase with an effective amount or concentration of any of:

(a) a compound of formula (IA) or (IB)

[chemical structure]

wherein R is hydrogen, alkyl, cycloalkyl, heterocyclyl, aryl, or heteroaryl, wherein any alkyl, cycloalkyl, heterocyclyl, aryl, or heteroaryl is substituted with 0-4 J;

the ring comprising X¹-X⁴ is present or absent; when absent, the ring comprising Y¹ and Y² is further substituted with R; when present, each of X¹-X⁴ is an independently selected C or N, provided that when any of X¹-X⁴ is N, the respective R¹-R⁴ is absent;

each of R¹-R⁴, when present, is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

W is O or S;

Y¹ and Y² are independently N, O, S, or CR;

each of Z¹-Z³ is an independently selected CR⁵, CR⁵═CR⁵, N, or N═N;

each R⁵ is independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof; or,

(b) a compound of formula (II)

[chemical structure]

wherein each of X¹-X⁴ is an independently selected C or N, provided that when any of X¹-X⁴ is N, the respective R¹-R⁴ is absent; when present, each of R¹-R⁴ is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

W¹ is O, S, or CH₂;

W² is O or S;

Y¹ and Y² are independently selected O, S, or NR;

each R is independently selected hydrogen or alkyl;

Ar is aryl or heteroaryl, substituted with 0-4 J;

n is 1 to about 6;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′; C(O)N(R′)₂, OC(O)N(R′)₂, C(S)N(R′)₂, (CH₂)₀₋₂N(R′)C(O)R′, (CH₂)₀₋₂N(R)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof; or,

(c) a compound of formula (III)

[chemical structure]

wherein each of X¹-X⁴ is an independently selected C or N, provided that when any of X¹-X⁴ is N, the respective R¹-R⁴ is absent; when present, each of R¹-R⁴ is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;

Y is O or S;

R⁵ is alkyl, cycloalkyl, aryl, heterocyclyl or heteroaryl, substituted with 0-4 J;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof; or,

(d) a compound of the formula (IV)

[chemical structure]

wherein X¹ and X² are each independently C or N, provided that when X¹ or X² is N, the respective R¹ or R² is absent; when present, R¹ and R² each is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J; or R¹ and R² together with X″ and X² form an optionally substituted cycloalkyl, heterocyclyl, aryl or heteroaryl ring;

W is O, S, or CH₂;

Y is O or S;

Z is absent or is NR;

each R is independently selected H or alkyl;

n1 and n2 are each independently 0 to 6;

Ar is aryl or heteroaryl, substituted with 0-4 J;

J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof; or,

(e) a compound of formula (V)

[chemical structure]

wherein each independently selected R, R⁵, Y¹ and Y², are as defined for the compound of formula (I); or any salt thereof; or,

(f) a compound of formula (VI)

[chemical structure]

wherein R⁵ is as defined for the compound of formula (I), and R⁶ is hydrogen, alkylcarbonyl, cycloalkylcarbonyl, aroyl, or heteroaroyl; or a salt thereof.

(g) a compound of formula (VII)

[chemical structure]

wherein R, R¹-R⁴, Y and Y², are as defined as for the compound of formula (I), and R² comprises OH, OR, or N(R)R⁸, wherein R⁸ is aminoalkyl, mono- or di-alkylaminoalkyl, heterocyclylalkyl, or heteroarylalkyl; or a salt thereof; or,

(h) any of the following compounds:

[chemical structure]

[chemical structure]

[chemical structure]

[chemical structure]

[chemical structure]

[chemical structure]

[chemical structure]

[chemical structure]

[chemical structure]

or any salt thereof.

In various embodiments the transfection-enhancing compound used to inhibit a cytosine deaminase, such as a mammalian DNA polynucleotide cytosine deaminase, to enhance or boost transfection of a target cell with a foreign DNA, or to treat a malcondition comprising a genetic disorder treatable by genetic therapy, can be a catechol derivative, i.e., an ortho-hydroxyphenol derivative, or the transfection-enhancing compound can be a thioimidazole derivative, such as a thiobenzimidazole derivative, or a thiazole derivative, or a thiadiazole derivative.

In various embodiments, the invention provides methods of increasing or enhancing the efficiency or fidelity of DNA transfection of mammalian cells by foreign DNA, such as plasmid DNA, etc., which can include genetically engineered sequences of various types, the method comprising contacting the mammalian cell, or any other kind of eukaryotic cell, with the foreign DNA, under conditions suitable for transfection or transduction to occur, such as the plasmid DNA, in the presence of an effective cytosine deaminase inhibitory amount of any of the above-listed cytosine deaminase inhibitory compounds. In various embodiments, the invention provides a method to improve the transfection efficiency of mammalian cells, either in vivo or in vitro, wherein the cells are engineered with a greater efficiency and fidelity, i.e., a higher transfection rate, compared to art methods. The foreign DNA can include single-stranded or double-stranded DNA fragments, plasmids, cosmids, synthetic chromosomes, engineered viral DNA, and the like. The contacting can be carried out using materials and methods known in the art as transfection or transduction adjuvants, such as cationic lipids, cationic polymers, cationic peptides, pegylated liposomes, electroporation, and the like.

In various embodiments, the invention provides a method of enhancing or stimulating transfection of a eukaryotic cell with foreign DNA, comprising contacting the cell with an effective amount of the foreign DNA, under conditions suitable for transfection or transduction to occur, in the presence of an effective amount or concentration of any of the transfection-enhancing cytosine deaminase inhibitors described above. The foreign DNA can be bacterial plasmid DNA containing an engineered DNA sequence, and the eukaryotic cell can be a mammalian cell, such as a human cell.

In various embodiments, the invention provides a method of treating a genetic disease in a patient afflicted therewith, the method comprising contacting a cell or tissue, in vivo in the body of a patient afflicted with the genetic disease, with a curative foreign DNA, under conditions suitable for transfection or transduction to occur, in the presence of an effective amount of any of the transfection-enhancing compounds of the invention, for example, wherein the curative foreign DNA comprises plasmid DNA, wherein the plasmid DNA incorporates an engineered DNA sequence or sequences wherein the engineered sequence or sequences isare adapted to code for correction of a genetic deficiency of the patient. Compounds of the invention include any of the compounds of formulas (IA), (IB), (II), (III), (IV), (V), (VI), or (VII) discussed above, or any of the compounds of the above discussion in section (h) thereof, not claimed as compounds of the invention, but disclosed and claimed for practice of the methods of the invention herein, including a a method of inhibiting a cytosine deaminase, a method of enhancing or stimulating transfection of a eukaryotic cell with foreign DNA, methods of increasing or enhancing the efficiency or fidelity of DNA transfection of mammalian cells by foreign DNA, and a method of treating a genetic disease in a patient afflicted therewith, as discussed in the preceding paragraphs.

Examples of genetic diseases whose treatments could be enhanced as described here include any blood disorders treatable by stem cell genetic engineering such as Fanconi anemia or beta-thalassemia (ie. enhancement of ex vivo gene delivery). It may also be applicable in the future to in vivo gene therapy to correct blood and solid tissue disorders.

In various embodiments, the invention identifies small molecules that potently and specifically inhibit A3 DNA deaminase activity in vitro and in vivo. A3 proteins irreversibly ‘modify’ foreign DNA by deaminating cytosines to uracils as shown in FIG. 1. Subsequent excision of the DNA U's by the uracil DNA glycosylase UNG2 results in foreign DNA degradation and clearance (28). Foreign DNA within a cell causes a signalling cascade that results in the expression of APOBEC3A. APOBEC3A catalyzes the deamination of cytosine to uracil within the foreign DNA molecule. These uracils are removed by a DNA repair enzyme called Uracil DNA-glycosylase (encoded by the gene UNG2), leaving gaps in the DNA sequence called abasic sites. These abasic sites are substrates for endonucleolytic cleavage by APEX and related enzymes. The ultimate result is the removal by degradation of the foreign DNA.

High-throughput screening of nearly 30,000 compounds has identified molecules that inhibit A3 DNA deaminase activity in vitro. MN132, MN137, MN141, and MN152 are a few exemplary molecules identified from high-throughput screening. A schematic representation of the SS-DNA cytidine deamination assay is shown in FIG. 2A. To screen for inhibitors of A3A or A3G DNA cytosine deaminase activity, a previously described fluorescence-based assay has been modified, optimized, and miniaturized (FIG. 2A) (30). C-to-U by A3A or A3G creates a substrate uracil that is excised by UDG, and the resulting abasic site is attacked by hydroxide to release 6-FAM fluorescence from the TAMRA quench. Recombinant A3A- or A3G-myc-His purified from a HEK-293-based cell line is incubated with uracil DNA glycosylase (UDG), 10 μM library compound (or a DMSO control), and a cytosine-containing, single-stranded DNA substrate attached to a 5′ 6-FAM fluorescent tag and a 3′ TAMRA quench molecule. During a 2 hr incubation period, the A3 protein deaminates 5′-C-to-U and UDG excises the uracil to leave an abasic site. DNA cleavage by NaOH produces a fluorescent signal by releasing the 6-FAM fluorophore from the TAMRA quench. This assay is highly sensitive and reproducible (A3G Z score=0.86 and A3A Z score=0.88 on the University of Minnesota ITDD HTS platform).

Small molecules are considered inhibitors if they inhibit the reaction by 50% or more in HTS and reproduce in subsequent dose response tests. The representative A3A and A3G inhibition data from inhibitors of the invention is shown in FIG. 2B.

FIGS. 3A and 3B show data relating to foreign DNA restriction assay in living HEK-293 cells. Secondary cell-based screens revealed that some of these compounds function in human cells to boost transfection efficiencies. Compounds were dissolved in DMSO, and tested in vitro, to determine if the efficiency of transient GFP expression from a plasmid is improved in the presence of A3A as illustrated in FIG. 3A. A3A deaminates transfected DNA and marks it for UNG2-dependent clearance (28). In a transient transfection experiment, this equates to a rapid acceleration of the rate of GFP decay from day 1 to day 5 post-transfection. The restriction ratios of A3A-E72A catalytic mutant and A3A are compared in FIG. 3B.

HEK-293T cells are transfected with plasmids expressing GFP and either catalytically active APOBEC3A or the catalytically inactive E72A variant as a negative control. These transfected cells are split into wells containing various inhibitors or DMSO. On day 1 and day 5 post-transfection, the percentage of cells that are GFP positive is measured by flow cytometry. The restriction ratio is the percentage of GFP positive cells on day 5 divided by the percentage of GFP positive cells on day 1. True inhibitors should increase the restriction ratio. In this assay, about 30% of cells transfected with A3A-E72A and GFP on day 1 are still positive on day 5 compared to only about 5% of cells transfected with catalytically active A3A and GFP (DMSO control) as shown in FIG. 3B. A number of inhibitors have no effect on the restriction ratio. Some result in a moderate increase in the restriction ratio and have an intermediate chemotype while a few dramatically increase the restriction ratio to levels approaching that of the catalytic mutant. The asterisk indicates that these results are significant at the 95% confidence level. Error bars indicate standard deviation from three independent assays.

Representative DNA deaminase enzyme targets were obtained as shown in FIGS. 4A to 4D and as described in the literature (28, 30). Compounds of the invention can have an IC50 value for enzymes such as A3A or A3G in the micromolar to nanomolar concentration range.

Some DNA deaminase inhibitors are predicted to potently block the activity of all seven A3 family members and improve transfection efficiencies in all types of human cells (i.e., broad spectrum inhibition). An example of such an inhibitor would be an active site competitor because the active sites are highly conserved all bearing a defining motif: H-x₁-E-x₂₃₋₂₈-C-x₂₋₄-C (x can be nearly any one of the twenty amino acids) (35, 36). The feasibility of broad-spectrum inhibition is supported by the data showing that many of the current inhibitors block both A3A and A3G activity and by the fact that all members of this family share sequence- and structurally-conserved zinc coordinating active site (2, 26). The overall approach for achieving this goal is to (i) perform parallel HIS using A3A and A3G to identify candidate broad-spectrum inhibitors, (ii) construct novel sub-libraries to improve desirable properties of candidate broad spectrum inhibitors, and (iii) implement targeted secondary screens to identify A3AG inhibitors that also block the deaminase activity of the other five A3s (A3BCDEFH). Here, this final assay can be addressed by four secondary screens, which are presented from least complex (in vitro assays with recombinant proteins) to most complex (primary multi-A3+ human cells ex vivo).

Protocols were developed for purification of A3-myc-His proteins from HEK-293 cells. FIG. 4A and FIG. 4B illustrate representative human A3A-myc-His proteins prepared from human cells. The hexa-histidine tag (His) enables purification and the myc tag is used for protein identification by immunoblotting. High (80-90%) purity is achieved routinely, and all proteins show robust activity in our fluorescence-based C-to-U deaminase assay as shown in FIGS. 4C and 4D.

All newly synthesized compounds can be tested in dose response experiments against recombinant A3A and A3G to identify the sub-library_compounds that have dual-inhibitory activity like the parental molecules. The dose responses for the compounds of the invention against A3A or A3G are shown in FIG. 2B. Most compounds can be regarded as specific because they do not block the only other enzyme in the assay system UDG, the lone exception being ATA, a non-specific nucleic acid enzyme inhibitor. The data of control assay to verify that inhibitors do not affect E. Coli uracil-DNA glycosylase, with ATA served as a positive control is shown in FIG. 2C.

These initial experiments can also identify the subset with improved IC50 values. Second, all confirmed inhibitors can be tested against the other five recombinant human A3 proteins (A3BCDEFH) to identify broader-spectrum inhibitors. Third, reactions can be performed with U-containing oligos and UDG (i.e., without A3s) to re-confirm that all new inhibitors have not become general enzyme inhibitors. These assays can be carried out in 96 or 384 well plate formats. Some of these molecules identified can also be broad-spectrum inhibitors as shown in FIG. 5. HEK-293T cells were transfected with an A3A expression plasmid, and total cell extracts were used in DNA deaminase assays. Non-transfected or A3A-E72A catalytic mutant transfected cell extracts show no activity (28).

As a pre-requisite to testing compounds in living cells, we will take advantage of prior observations demonstrating activity of individual over-expressed A3s in HEK-293 cell extracts (HEK-293 cells are A3-deficient) (28, 30). It is hypothesized that compounds that retain inhibitory activity in a cellular extract will have a better chance of eliciting activity in living cells. Like the recombinant protein assay described above, assays with A3-containing cell extracts are done in 96 and 384 well formats and the read-out is relative fluorescence units. Representative data with exemplary compounds MN132 and MN141 are shown in FIG. 5. The higher IC50 values in cell extracts may be due to a wide variety of factors. Nevertheless, positive results with this simple assay provide an important stepping-stone toward identifying compounds that will be more likely to elicit activity in living cells.

The GFP-based assay is described in FIG. 3A for A3A. Since all molecules from the aforementioned assays will potently inhibit A3A, A3G, and possibly other A3s, A3A is used as an in vivo model for foreign DNA restriction. In these experiments, HEK-293 cells will be transiently transfected with an A3A expression plasmid and a GFP reporter plasmid using standard lipid-based reagents (e.g., FuGene, Roche). An A3A-E72A catalytic mutant serves as the negative control. In contrast to this mutant or the vector only reactions, which typically yield over 25% fluorescent cells, A3A causes a rapid loss of transient GFP expression (e.g., compare day 3 bars in FIG. 6A). Expression of A3A results in rapid decay of GFP expression over time while the A3A catalytic mutant (E72A) and vector control show similar decay. Error bars are the standard deviation from three independent transfections. Molecules that inhibit the activity of exogenously expressed A3A are predicted to preserve GFP fluorescence (e.g., representative data in FIG. 3B and idealized schematic in FIG. 6B). In FIG. 6B, on the left, in the presence of DMSO, A3A expression results in degradation of the GFP plasmid and on the right, a potent inhibitor of A3A results in increased expression of GFP in the presence of A3A. Inhibitors of A3A phenocopy the catalytic mutant resulting in slower expression decay and higher stability of the plasmid itself.

Compounds that inhibit A3A can also be tested against the other six human A3s using this GFP-based assay. An advantage of this approach is that it has already been adapted to 96 well plate format and high-throughput flow cytometry. A second advantage is that the flow cytometry dot plots (cell profiles) enable us to simultaneously assess cytotoxicity. Each experiment will be done in triplicate with a wide range of compound concentrations to ensure that both inhibitory activity and cellular toxicity (at some point almost all chemicals become toxic) can be documented.

Once cell-compatible A3 inhibitors are identified, they can be tested in cell lines and primary cell types where qPCR is used to define the expressed endogenous A3 repertoire (23). For instance, the T cell line CEM expresses high levels of five A3 mRNAs and it is difficult to transfect. It can be predicted that a complete inhibition of endogenous A3 activity will render this line more transfectable as described above. In contrast, the inhibitors should not alter the transfection efficiency of A3-deficient cell lines such as SupT11 and possibly HEK-293. Primary CD14+ monocytes and macrophages are notoriously difficult to transfect, and it can be hypothesized that this is largely due to high levels of A3A and the other A3s (28).

Primary human monocytes were purified by negative selection and nucleofected with a GFP expression vector. Since these cells naturally express A3A, transfection with A3A expression plasmids was not necessary. The pool of transfected cells was split into wells containing media plus vehicle control or MN132. The percentage of cells expressing GFP was determined 3 days post-transfection by flow cytometry as shown in FIG. 7A. A variety of different cell lines (lymphoid, liver, breast, brain, epithelial, and colorectal origins) and distinct primary white blood cell types (T, B, monocyte, neutrophil, etc.) can be used in these experiments to test the central hypothesis that the endogenous A3s constitute a general barrier to foreign DNA and genetic engineering. DMSO treated primary monocytes were not detectably transfected but cells treated with MN132 exhibited dose dependent increases in the percentage of cells expressing GFP as shown in FIG. 7B.

FIGS. 8A-8D illustrate inhibition of foreign DNA restriction by compounds of the invention. As described above, HEK 293T cells were transfected with GFP and either A3A or A3A catalytic mutant (E72A) plasmids. Flow cytometry was done to determine transfection efficiency on day 1 and again on day 5. The restriction ratio is the ratio of the percentage of cells that retain GFP expression on day 5 compared to day 1. Error bars are the standard deviation of three independent assays.

### Experimental Details for Formulas I-VII

[chemical structure]

Isatin (1.93 g, 13.12 mmol) was suspended in 50 mL of ethanol and heated to 60° C. at which point a solution of potassium hydroxide (33% w/v, 7.4 mL) was added and stirred at 60° C. for 15 minutes. 2-Acetyl-5-methylfuran (1.8 mL, 15.4 mmol) was added dropwise to heated solution. Upon complete addition, reaction solution was further warmed to reflux. After 48 hours, reaction solution was concentrated to a dark brown solid. A solution (˜60 mL) of 20% acetic acid in water was added slowly to adjust the pH to 5. The precipitate that formed was vacuum filtered and washed with H₂O (50 mL) and then hexanes (100 mL). The yellow solid was dried via high vac to afford 1.87 g of 2-(5-methylfuran-2-yl)quinoline-4-carboxylic acid.

[chemical structure]

To a stirring suspension of acid (2.97 g, 24.12 mmol) and thiosemicarbazide (2.18 g, 23.92 mmol) in dioxane (16 mL) at room temperature under nitrogen was slowly added POCl₃ (2.7 mL). The suspension was warmed to reflux at which time everything went into solution. After three hours, there was a precipitate forming in the flask. Reaction mixture was cooled to room temperature and poured into 30 mL of ice water. A solution of 50% aqueous NaOH was used to adjust the pH to 9. The orange precipitate was vacuum filtered and dried in vacuo to afford 1.23 g (28%) of crude 5-(pyridin-4-yl)-1,3,4-thiadiazol-2-amine.

[chemical structure]

To a 0° C. solution of the quinoline carboxylic acid (101 mg, 0.399 mmol) and the aminothiodiazole (79 mg, 0.443 mmol) in DMF (4.0 mL) under nitrogen was added EDCI-HCl (86 mg, 0.448 mmol) and HOBt (82 mg, 0.607 mmol) followed by 240 mL of N-methylmorpholine. The reaction solution was slowly warmed to room temperature. After stirring for 36 hours, the reaction solution was poured into 20 mL of saturated aqueous ammonium chloride and extracted with EtOAc (3×15 mL). The organic layer was washed with brine (20 mL), water (20 mL), dried over Na₂SO₄, filtered and concentrated. The crude solid was purified on a Combiflash Rf using SiO₂ with DCMMeOH to afford 36 mg (22%) of 2-(5-methylfuran-2-yl)-N-(5-(pyridin-4-yl)-1,3,4-thiadiazol-2-yl)quinoline-4-carboxamide as an yellow solid.

[chemical structure]

Isatin (2.01 g, 13.66 mmol) was suspended in 55 mL of ethanol and heated to 65° C. to dissolve the solid. A solution of potassium hydroxide (33% w/v, 6.7 mL) was added and stirred at 65° C. for 15 minutes. Acetophenone (1.8 mL, 15.4 mmol) was added dropwise to heated solution. Upon complete addition, reaction solution was warmed to reflux. After 48 hours, reaction solution was concentrated to a dark brown solid. A solution (˜60 mL) of 20% acetic acid in water was added slowly to adjust the pH to 5. The precipitate that formed was vacuum filtered and washed with EtOH (˜30 mL) and hexanes (˜30 mL). The beige solid was dried via high vac to afford 2.02 g (60%) of 2-phenylquinoline-4-carboxylic acid.

[chemical structure]

To a mixture of acid (2.23 g, 11.95 mmol) and thiosemicarbazide (1.10 g, 12.07 mmol) at 0° C. under nitrogen was added slowly 6.6 mL of POCl₃. Reaction mixture stirred for 30 minutes at 0° C. and then heated to reflux. After 3 hours, reaction solution was cooled to room temperature and poured into 25 mL of ice water. The pH of the suspension was adjusted to 9 with 50%-aqueous NaOH solution. Precipitate was filtered and washed with H₂O and dried via high vac to afford 2.65 g (95%) of 5-((4-chlorophenoxy)methyl)-1,3,4-thiadiazol-2-amine as a beige solid.

[chemical structure]

To a 0° C. solution of the carboxylic acid (251 mg, 1.007 mmol) and the aminothiodiazole (268 mg, 1.109 mmol) in DMF (10 mL) under nitrogen was added EDCI-HCl (214 mg, 1.116 mmol) and HOBt (206 mg, 1.524 mmol) followed by 670 mL of N-methylmorpholine. The reaction solution was slowly warmed to room temperature. After stirring for 36 hours, the reaction solution was poured into 20 mL of saturated aqueous ammonium chloride and extracted with EtOAc (3×15 mL). The organic layer was washed with brine (20 mL), water (20 mL), dried over Na₂SO₄, filtered and concentrated. The crude solid was purified on a Combiflash Rf using SiO₂ with DCMMeOH to afford 221 mg (46%) of N-(5-((4-chlorophenoxy)methyl)-1,3,4-thiadiazol-2-yl)-2-phenylquinoline-4-carboxamide as an off-white solid.

[chemical structure]

A solution of thionyl chloride (2.0 mL, 27.4 mmol) and iodobenzoic acid (192 mg, 0.691 mmol) was heated to reflux under nitrogen. After 3 hours, the reaction solution was cooled to room temperature and concentrated to a colorless oil. The colorless oil was dissolved in p-xylenes (7.0 mL). Pyridine (60 mL, 0.75 mmol) and 2-aminothiophenol (75 mL, 0.69 mmol) were then added and the solution was stirred at room temperature under nitrogen. A pale yellow precipitate formed in the flask. After 2 hours, p-toluenesulfonic acid hydrate (657 mg, 3.454 mmol) was added and the reaction mixture was heated to reflux. After 18 hours, the reaction mixture was cooled to room temperature and poured into EtOAc (30 mL) and washed with brine (2×20 mL) and water (30 mL). The organic layer was dried over Na₂SO₄, filtered and concentrated. The crude oil was purified via Combiflash Rf on SiO₂ using hexanes/EtOAc to afford 192 mg (70%) of 2-(3-iodo-4-methoxyphenyl)benzo[d]thiazole as a colorless solid.

[chemical structure]

PyBop (675 mg, 1.297 mmol) and DIPEA (0.3 mL) was added to a 0° C. solution of diamine (127 mg, 1.174 mmol) and acid (360 mg, 1.295 mmol) in DMF (3.0 mL) under nitrogen. The solution was slowly warmed to room temperature. After 24 hours, the reaction solution was poured into saturated aqueous NaHCO₃(20 mL) and extracted with EtOAc (2×20 mL). The organic layer was washed with brine (2×20 mL), dried over MgSO₄, filtered and concentrated. The crude solid was purified on a Combiflash Rf using SiO₂ with CH₂Cl₂MeOH to afford 367 mg (85%) of amide product as a white solid.

A suspension of the amide (294 mg, 0.779 mmol) in AcOH (8.0 mL) was heated to reflux. After 24 hours, the solution was cooled to room temperature and concentrated in vacuo to a tan solid. The crude solid was purified via Combiflash Rf using SiO₂ with CH₂Cl₂MeOH to afford 2-(3-iodo-4-methoxyphenyl)-1H-benzo[d]imidazole (193 mg, 69%) as a colorless solid.

[chemical structure]

A mixture of diamine (3.04 g, 28.11 mmol) and potassium ethylxanthate (6.79 g, 42.36 mmol) in EtOH (30 mL) and H₂O (4.5 mL) was heated to reflux at which time all solid dissolved. After 3 hours, decolorizing carbon (5 g) was added and reaction was heated for an additional 15 minutes. Warm reaction mixture was filtered through celite, followed by hot water (30 mL). The filtrate was warmed to 65° C. and a 20% solution of AcOH in H₂O (10 mL) was added slowly to heating solution. Reaction solution foamed some and a precipitate formed with addition. Upon complete addition the reaction mixture was cooled to room temperature, capped and allowed to stand at 4° C. After 3 hours, the resulting white precipitate was vacuum filtered and rinsed with 30 mL of H₂O. The white solid was dried via high vac to afford 3.54 g (84%) of 2-mercaptobenzimidazole.

[chemical structure]

To a 0° C. solution of bromoacetylbromide (330 mL, 3.78 mmol) in dry THF (6.0 mL) under nitrogen was slowly added a solution of aniline (468 mg, 2.515 mmol) and DMAP (156 mg, 1.277 mmol) in THF (6.0 mL). The reaction mixture was stirred at 0° C. for 1 hour, then room temperature for 1 hour. The reaction solution was poured into 30 mL of water and extracted with CH₂Cl₂ (2×30 mL). The organic layer was washed with H₂O (2×30 mL), dried over MgSO₄; filtered and concentrated. The crude solid was purified via Combiflash Rf on SiO₂ with hexanes/EtOAc to afford 662 mg (86%) of 2-bromo-N-(4-bromo-3-methylphenyl)acetamide as a colorless solid.

[chemical structure]

A suspension of 2-mercaptobenzimidazole (323 mg, 2.150 mmol), bromoacetamide (728 mg, 2.387 mmol) and K₂CO₃ (448 mg, 3.241 mmol) in acetone (22 mL) was stirred at room temperature under nitrogen. After 24 hours, the reaction mixture was poured into 30 mL of saturated aqueous NH₄Cl and extracted with CH₂Cl₂ (2×30 mL). The organic layer was washed with H₂O (30 mL), dried over Mg SO₄, filtered and concentrated. The crude solid was purified via Combiflash Rf on SiO₂ with hexanes/EtOAc to afford 387 mg (48%) of 2-((1H-benzo[d]imidazol-2-yl)thio)-N-(4-bromo-3-methylphenyl)acetamide as a colorless solid.

[chemical structure]

To a suspension of NH₄Cl (631 mg, 11.799 mmol) in anhydrous toluene (12 mL) at 0° C. under nitrogen was slowly added Al(CH₃)₃ solution (5.9 mL, 2.0M in toluene) over 5 minutes. Suspension stirred at 0° C. for 5 minutes and then slowly warmed to room temperature, stirring an additional 30 minutes at room temp. To this stirring solution was then added 4-methoxybenzonitrile (1.57 g, 11.79 mmol) in 2.5 mL of toluene. The reaction was heated to 80° C. After 18 hours, the reaction solution was cooled to room temperature and was carefully poured into a slurry of SiO₂ (25 g) in CHCl₃ (60 mL). After stirring for 10 minutes, the mixture was filtered and the filter cake was rinsed with MeOH (50 mL). The filtrate was concentrated to give 1.43 g (81%) of benzimidamide as a white solid, which was used without further purification.

A suspension of benzimidamide (459 mg, 3.056 mmol), carbondisulfide (460 mL, 7.62 mmol) and sulfur (121 mg, 3.774 mmol) in MeOH (3.8 mL) was stirred under nitrogen at room temperature. To this was slowly added 1.9 mL of sodium methoxide solution (25% w/v in MeOH). Upon complete addition, solution was heated to reflux. After 24 hours, the reaction solution was cooled to room temperature and the pH of the solution was adjusted to 4 with aqueous HCl (1M). The yellow precipitate that formed was filtered and dried to afford 249 mg (36%) of 3-(4-methoxyphenyl)-1,2,4-thiadiazole-5-thiol, which was used without further purification.

[chemical structure]

To a 0° C. solution of furfurylamine (100 mL, 1.13 mmol) and DMAP (67 mg, 0.55 mmol) in dry THF (8 mL) under nitrogen was slowly added bromoacetyl bromide (150 mL, 1.72 mmol). The reaction solution was slowly warmed to room temperature. After 18 hours, the reaction solution was poured into H₂O (20 mL) and extracted with EtOAc (2×20 mL). The organic layer was washed with NaHCO₃ (sat aqueous, 20 mL), dried via Na₂SO₄, filtered and concentrated. The resulting crude solid was purified via Combiflash Rf on SiO₂ using hexanes/EtOAc to afford 147 mg (60%) of 2-bromoacetamide as a white solid.

[chemical structure]

A suspension of 1,2,4-thiadiazole-5-thiol (90 mg, 0.401 mmol), 2-bromoacetamide (96 mg, 0.440 mmol) and K₂CO₃ (85 mg, 0.615 mmol) in acetone (4.0 mL) was stirred at room temperature under nitrogen. After 24 hours, the reaction mixture was poured into H₂O (30 mL) and extracted with EtOAc (2×30 mL). The organic layer was washed with sat aqueous NH₄Cl (20 mL), H₂O (30 mL), dried over MgSO₄, filtered and concentrated. The resulting crude solid was purified via Combiflash Rf on SiO₂ using hexanes/EtOAc to afford 76 mg (53%) of N-(furan-2-ylmethyl)-24(3-(4-methoxyphenyl)-1,2,4-thiadiazol-5-yl)thio)acetamide as a white solid.

[chemical structure]

A solution of thiadiazol-2-amine (250 mg, 1.03 mmol) and pyridine (190 mL, 2.35 mmol) in CH₂Cl₂ was cooled to 0° C. under nitrogen. Acetylchloride (90 mL, 1.27 mmol) was added slowly. The reaction solution was slowly warmed to room temperature. After 24 hours, the reaction solution was poured into brine (20 mL) and extracted with CH₂Cl₂ (2×20 mL). The organic layer was washed with H₂O (30 mL), dried over Na₂SO₄, filtered and concentrated. The resulting solid was purified via Combiflash Rf on SiO₂ using hexanes/EtOAc to afford 181 mg (62%) of N-(5((4-chlorophenoxy)methyl)-1,3,4-thiadiazol-2-yl)acetamide as a biege solid.

[chemical structure]

To a 0° C. solution of 2-(5-methylfuran-2-yl)quinoline-4-carboxylic acid (174 mg, 0.687 mmol) in DMF (6.8 mL) under nitrogen was added EDCI-HCl (146 mg, 0.762 mmol) and HOBt hydrate (141 mg, 1.043 mmol), followed by N-methylmorpholine (450 mL) and N,N-dimethylethylenediamine (85 mL, 0.778 mmol). The reaction solution was slowly warmed to room temperature. After 4 hours, saturated aqueous NH₄Cl (10 mL) was added. The reaction mixture was poured into 20 mL of EtOAc and 10 mL of H₂O. The aqueous layer was separated and washed with EtOAc (2×20 mL). The combined organic layers were washed with brine (20 mL), dried over Na₂SO₄, filtered and concentrated. The crude solid was purified via Combiflash Rf on SiO₂ using hexanes/EtOAc to afford 133 mg (60%) of N-(2-(dimethylamino)ethyl)-2-(5-methylfuran-2-yl)quinoline-4-carboxamide as an off-white solid.

### EXAMPLES

### DNA Cytidine Deaminase Activity Assays

PBMC or transfected HEK-293T cell lysates were prepared as above for immunoblotting. The deaminase activity in the lysates was determined using a FRET-based assay essentially as described⁵⁹. Briefly, serial dilutions of lysates were incubated for 2 h at 37° C. with a DNA oligonucleotide 5′-(6-FAM)-AAA-TTC-TAA-TAG-ATA-ATG-TGA-(TAMRA). FRET occurs between the fluorophores, decreasing FAM fluorescence. If cytidine deaminase activity is present in the lysates, the single cytidine is converted to uridine, which is then excised by uracil DNA glycosylase (NEB). Resulting abasic sites are cleaved by incubating reactions for 2 min at 95° C. Once cleaved, the FAM and TAMRA labels are physically separated, FRET diminishes, and FAM fluorescence increases. Fluorescence is measured on the Lightcycler 480 instrument (Roche). See also: Stenglein, M. D., M. B. Burns, M. Li, J. Lengyel, and R. S. Harris. 2010. APOBEC3 proteins mediate the clearance of foreign DNA from human cells. Nat Struct Mol Biol 17:222-9, incorporated by reference herein.

### DOCUMENTS CITED

1. Albin, J. S., and R. S. Harris. 2010. Interactions of host APOBEC3 restriction factors with HIV-1 in vivo: implications for therapeutics. Expert Rev Mol Med 12:e4.
2. Chen, K. M., E. Harjes, P. J. Gross, A. Fahmy, Y. Lu, K. Shindo, R. S. Harris, and H. Matsuo. 2008. Structure of the DNA deaminase domain of the HIV-1 restriction factor APOBEC3G. Nature 452:116-9.
3. Flintoft, L. 2010. Human cells clear foreign DNA. Nature Reviews Genetics 11:172.
4. Ghodsi, R., A. Zarghi, B. Daraei, and M. Hedayati. 2010. Design, synthesis and biological evaluation of new 2,3-diarylquinoline derivatives as selective cyclooxygenase-2 inhibitors. Bioorganic & Medicinal Chemistry 18:1029-1033.
5. Hargrave, K. D., F. K. Hess, and J. T. Oliver. 1983. N-(4-Substituted-Thiazoly)oxamic acid derivatives, a new series of potent, orally active antiallergy agents. Journal of Medicinal Chemistry 26:1158-1163.
6. Harris, R. S., K. N. Bishop, A. M. Sheehy, H. M. Craig, S. K. Petersen-Mahrt, I. N. Watt, M. S. Neuberger, and M. H. Malim. 2003. DNA deamination mediates innate immunity to retroviral infection. Cell 113:803-9.
7. Harris, R. S., S. K. Petersen-Mahrt, and M. S, Neuberger. 2002. RNA editing enzyme APOBEC1 and some of its homologs can act as DNA mutators. Mol Cell 10:1247-53.
8. Hultquist, J. F., and R. S. Harris. 2009. Leveraging APOBEC3 proteins to alter the HIV mutation rate and combat AIDS. Future Virology 4:605-619.
9. Jaen, J. C., L. D. Wise, B. W. Caprathe, H. Tecle, S. Bergmeier, C. C. Humblet, T. G. Heffner, L. T. Meltzer, and T. A. Pugsley. 1990. 4-(1,2,5,6-Tetrahydro-1-alkyl-3-pyridinyl)-2-thiazolamines: A novel class of compounds with central dopamine agonist properties. Journal of Medicinal Chemistry 33:311-317.
10. Kidd, J. M., T. L. Newman, E. Tuzun, R. Kaul, and E. E. Eichler. 2007. Population stratification of a common APOBEC gene deletion polymorphism. PLoS Genet. 3:e63.
11. Kitagawa, H., T. Ozawa, S. Takahata, and M. Iida. 2007. Phenylimidazole derivatives as new inhibitors of bacterial enoyl-ACP reductase FabK. Bioorganic & Medicinal Chemistry Letters 17:4982-4986.
12. Koparir, M., A. Cansiz, A. Cetin, and C. Kazaz. 2005. Synthesis of (1R,2R)-1,2-bis-(5-(4-hydroxynaphthalen-1-ylazo) 1,3,4thiadiazol-2-yl)-ethane-1,2-diol. Chemistry of Natural Compounds 41:569-571.
13. Lacasse, G., and J. M. Muchowski. 1973. Five-membered Heterocyclic Thiones. Part IV. 1,2,4-Thiadiazole-3-thiolates and 1,2,4-thiadiasolve5-thione. Canadien Journal of Chemistry 51:2353-2356.
14. Leung-Toung, R., J. Wodzinska, W. R. Li, J. Lowrie, R. Kukreja, D. Desilets, K. Karimian, and T. F. Tam. 2003. 1,2,4-thiadiazole: A novel Cathepsin B inhibitor. Bioorganic & Medicinal Chemistry 11:5529-5537.
15. Li, Z., J. L. Yu, J. Y. Yang, W. Zhu, Y. L. Zhao, Y. L. Xing, and X. C. Wang. 2006. Solvent-free synthesis of 2-amino-5-aryloxymenthyl-1,3,4-thiadiazoles and their coumarin or benzofuran bis-heterocyclic derivatives. Phosphorus Sulfur and Silicon and the Related Elements 181:183-190.
16. Medzhitov, R. 2007. Recognition of microorganisms and activation of the immune response. Nature 449:819-26.
17. Mikl, M. C., I. N. Watt, M. Lu, W. Reik, S. L. Davies, M. S, Neuberger, and C. Rada. 2005. Mice deficient in APOBEC2 and APOBEC3. Mol Cell Biol 25:7270-7.
18. Miyaura, N., and A. Suzuki. 1995. Palladium-catalyzed cross-coupling reactions of organoboron compounds. Chemical Reviews 95:2457-2483.
19. Muramatsu, M., K. Kinoshita, S. Fagarasan, S. Yamada, Y. Shinkai, and T. Honjo. 2000. Class switch recombination and hypermutation require activation-induced cytidine deaminase (AID), a potential RNA editing enzyme. Cell 102:553-63.
20. Nakamuta, M., B. H. Chang, E. Zsigmond, K. Kobayashi, H. Lei, B. Y. Ishida, K. Oka, E. Li, and L. Chan. 1996. Complete phenotypic characterization of apobec-1 knockout mice with a wild-type genetic background and a human apolipoprotein B transgenic background, and restoration of apolipoprotein B mRNA editing by somatic gene transfer of Apobec-1. J Biol Chem 271:25981-8.
21. Okeoma, C. M., N. Lovsin, B. M. Peterlin, and S. R. Ross. 2007. APOBEC3 inhibits mouse mammary tumour virus replication in vivo. Nature 445:927-30.
22. Okolotowicz, K. J., R. X. Shi, X. Y. Zheng, M. MacDonald, J. C. Reed, and J. R. Cashman. 2010. Selective benzimidazole inhibitors of the antigen receptor-mediated NF-kappa B activation pathway. Bioorganic & Medicinal Chemistry 18:1918-1924.
23. Refsland, E. W., M. D. Stenglein, K. Shindo, J. S. Albin, W. L. Brown, and R. S. Harris. 2010. Quantitative profiling of the full APOBEC3 mRNA repertoire in lymphocytes and tissues: implications for HIV-1 restriction. Nucleic Acids Research, 38, 4274-84.
24. Revy, P., T. Muto, Y. Levy, F. Geissmann, A. Plebani, O, Sanal, N. Catalan, M. Forveille, R. Dufourcq-Labelouse, A. Gennery, I. Tezcan, F. Ersoy, H. Kayserili, A. G. Ugazio, N. Brousse, M. Muramatsu, L. D. Notarangelo, K. Kinoshita, T. Honjo, A. Fischer, and A. Durandy. 2000. Activation-induced cytidine deaminase (AID) deficiency causes the autosomal recessive form of the Hyper-IgM syndrome (HIGM2). Cell 102:565-75.
25. Sehon, C. A., G. Z. Wang, A. Q. Viet, et al. 2008. Potent, Selective and Orally Bioavailable Dihydropyrimidine Inhibitors of Rho Kinase (ROCK1) as Potential Therapeutic Agents for Cardiovascular Diseases. Journal of Medicinal Chemistry 51:6631-6634.
26. Shandilya, S. M. D., M. N. L. Nalam, E. A. Nalivaika, P. J. Gross, J. C. Valesano, K. Shindo, M. Li, M. Munson, W. E. Royer, E. Harjes, T. Kouno, H. Matsuo, R. S. Harris, M. Somasundaran, and C. A. Schiffer. 2010. Crystal structure of the APOBEC3G catalytic domain reveals potential oligomerization interfaces. Structure 18:28-38.
27. Smith, J. L., W. Bu, R. C. Burdick, and V. K. Pathak. 2009. Multiple ways of targeting APOBEC3-virion infectivity factor interactions for anti-HIV-1 drug development. Trends Pharmacol Sci 30:638-46.
28. Stenglein, M. D., M. B. Burns, M. Li, J. Lengyel, and R. S. Harris. 2010. APOBEC3 proteins mediate the clearance of foreign DNA from human cells. Nat Struct Mol Biol 17:222-9.
29. Strebel, K., J. Luban, and K. T. Jeang. 2009. Human cellular restriction factors that target HIV-1 replication. BMC Med 7:48.
30. Thielen, B. K., K. C. Klein, L. W. Walker, M. Rieck, J. H. Buckner, G. W. Tomblingson, and J. R. Lingappa. 2007. T cells contain an RNase-insensitive inhibitor of APOBEC3G deaminase activity. PLoS Pathog 3:1320-34.
31. Tian, X., A. G. Switzer, S. A. Derose, et al. 2008. Discovery of orally bioavailable 1,3,4-trisubstituted 2-oxopiperazine-based melanocortin-4 receptor agonists as potential antiobesity agents. Journal of Medicinal Chemistry 51:6055-6066.
32. Wang, P., R. Wan, F. Han, and Y. Wang. 2009. One pot synthesis of 1,3,4-thiadiazole thiazolidinone derivatives. Journal of Chemical Research-S:671-673.
33. Yuye, Y. 2007. Microwave-assisted synthesis and biological activity of 2,5-disubstituted-1,3,4-thiadiazole. Asian Journal of Chemistry 19:3141-3144.
34. Zarghi, A., R. Ghodsi, E. Azizi, B. Daraie, M. Hedayati, and 0. G. Dadrass. 2009. Synthesis and biological evaluation of new 4-carboxylquinoline derivatives as cyclooxygenase-2 inhibitors. Bioorganic & Medicinal Chemistry 17:5312-5317.
35. (LaRue, R. S., S. R. Jónsson, K. A. T. Silverstein, M. Lajoie, D. Bertrand, N. El-Mabrouk, I. Hötzel, V. Andrésdóttir, T. P. L. Smith & R. S. Harris (2008). The artiodactyl APOBEC3 innate immune repertoire shows evidence for a multi-functional domain organization that existed in the ancestor of placental mammals. BMC Molecular Biology, 9, 104 (20 page Featured Article). 36. LaRue, R. S., V. Andresdottir, Y. Blanchard, S. G. Conticello, D. Derse, M. Emerman, W. C. Greene, S. R. Jônsson, N. R. Landau, M. Löchelt, H. S. Malik, M. H. Malim, C. Münk, S. J. O'Brien, V. K. Pathak, K. Strebel, S. Wain-Hobson, X.-F. Yu, N. Yuhki & R. S. Harris (2009), Guidelines for Naming Non-Primate APOBEC3 Genes and Proteins. Journal of Virology, 83, 494-497.

All patents and publications referred to herein are incorporated by reference herein to the same extent as if each individual publication was specifically and individually indicated to be incorporated by reference in its entirety.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A compound of formula (IA) or (IB) [chemical structure]
wherein R is hydrogen, alkyl, cycloalkyl, heterocyclyl, aryl, or heteroaryl, wherein any alkyl, cycloalkyl, heterocyclyl, aryl, or heteroaryl is substituted with 0-4 J;
the ring comprising X¹-X⁴ is present or absent; when absent, the ring comprising Y¹ and Y² is further substituted with R; when present, each of X¹-X⁴ is an independently selected C or N, provided that when any of X¹-X⁴ is N, the respective R¹-R⁴ is absent;
each of R¹-R⁴, when present, is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;
W is O or S;
Y¹ and Y² are independently N, O, S, or CR;
each of Z¹-Z³ is an independently selected CR⁵, CR⁵═CR⁵, N, or N═N;
each R⁵ is independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;
J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof;
provided the compound of formula (IA) or (IB) is not [chemical structure]
and provided that the compound of formula (IA) is not a compound that when Y¹ is N, Y² is CH, Z¹ is CR⁵, Z² and Z³ are both N, and R is phenyl, then R⁵ is hydrogen, halo, unsubstituted phenyl, unsubstituted furan-2-yl, unsubstituted pyridin-4-yl, or unsubstituted tetrahydrofuran-2-yl, or when R is hydrogen, then R⁵ is furan-3-yl, or pyridin-4-yl; or
a compound of formula (V) [chemical structure]
wherein R is hydrogen, alkyl, cycloalkyl, heterocyclyl, aryl, or heteroaryl, wherein any alkyl, cycloalkyl, heterocyclyl, aryl, or heteroaryl is substituted with 0-4 J;
each R⁵ is independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;
Y¹ and Y² are independently N, O, S, or CR;
J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′ SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof; or,
a compound of formula (VI) [chemical structure]
wherein R⁵ is as defined for the compound of formula (I), and R⁶ is hydrogen, alkylcarbonyl, cycloalkylcarbonyl, aroyl, or heteroaroyl; or any salt thereof; or,
a compound of formula (VII) [chemical structure]
wherein R is hydrogen, alkyl, cycloalkyl, heterocyclyl, aryl, or heteroaryl, wherein any alkyl, cycloalkyl, heterocyclyl, aryl, or heteroaryl is substituted with 0-4 J;
the ring comprising X¹-X⁴ is present or absent; when absent, the ring comprising Y¹ and Y² is further substituted with R; when present, each of X¹-X⁴ is an independently selected C or N, provided that when any of X¹-X⁴ is N, the respective R¹-R⁴ is absent;
each of R¹-R⁴, when present, is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;
Y¹ and Y² are independently N, O, S, or CR; and
R⁷ comprises OH, OR, or N(R)R⁸, wherein R⁸ is aminoalkyl, mono- or di-alkylaminoalkyl, heterocyclylalkyl, or heteroarylalkyl;
or any salt thereof.

2. The compound of formula (IA) of claim 1 comprising a compound of any of the following formulas [chemical structure]
wherein R and R⁵ are as defined in claim 1.

3. The compounds of claim 1 wherein the compound is any of the following: [chemical structure]
or a pharmaceutically acceptable salt thereof.

4.-10. (canceled)

11. The compound of claim 1, wherein the compound is [chemical structure]
or a pharmaceutically acceptable salt thereof.

12. (canceled)

13. The compound of claim 1, wherein the compound is any of the following [chemical structure]
or a pharmaceutically acceptable salt thereof.

14. (canceled)

15. The compound of claim 1, wherein the compound is any of the following [chemical structure] [chemical structure]
or a pharmaceutically acceptable salt thereof.

16. A method of inhibiting a DNA polynucleotide cytosine deaminase, comprising contacting the deaminase with an effective amount or concentration of any of:
(a) a compound of formula (IA) or (IB) [chemical structure]
wherein R is hydrogen, alkyl, cycloalkyl, heterocyclyl, aryl, or heteroaryl, wherein any alkyl, cycloalkyl, heterocyclyl, aryl, or heteroaryl is substituted with 0-4 J;
the ring comprising X¹-X⁴ is present or absent; when absent, the ring comprising Y¹ and Y² is further substituted with R; when present, each of X¹-X⁴ is an independently selected C or N, provided that when any of X¹-X⁴ is N, the respective R¹-R⁴ is absent;
each of R¹-R⁴, when present, is an independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;
W is O or S;
Y¹ and Y² are independently N, O, S, or CR;
each of Z¹-Z³ is an independently selected CR⁵, CR⁵═CR⁵, N, or N═N;
each R⁵ is independently selected hydrogen, alkyl, cycloalkyl, heterocyclyl, aryl, or heteroaryl, wherein any alkyl, cycloalkyl, heterocyclyl, aryl, or heteroaryl is substituted with 0-4 J;
J is F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further mono- or multi-substituted with J; or any salt thereof; or,
(e) a compound of formula (V) [chemical structure]
wherein each independently selected R, R⁵, Y¹ and Y², are as defined for the compound of formula (I); or any salt thereof; or,
(f) a compound of formula (VI) [chemical structure]
wherein R⁵ is as defined for the compound of formula (I), and R⁶ is hydrogen, alkylcarbonyl, cycloalkylcarbonyl, aroyl, or heteroaroyl; or a salt thereof; or,
(g) a compound of formula (VII) [chemical structure]
wherein R, X¹-X⁴, R¹-R⁴, Y¹, and Y², are as defined as for the compound of formula (I), and R⁷ comprises OH, OR, or N(R)R⁸, wherein R⁸ is aminoalkyl, mono- or di-alkylaminoalkyl, heterocyclylalkyl, or heteroarylalkyl; or a salt thereof; or,
(h) any of the following compounds: [chemical structure] [chemical structure]
or any salt thereof.

17. (canceled)

18. The method of claim 16 wherein the cytosine deaminase is any or all of APOBEC3A (A3A), APOBEC3B (A3B), APOBEC3C (A3C), APOBEC3D (A3D; also known as A3DE), APOBEC3F (A3F), APOBEC3G (A3G), APOBEC3H (A3H), AID, APOBEC1, APOBEC2, APOBEC4, or any of Z1, Z2, and/or Z3 type APOBEC3.

19. The method of claim 16 further comprising enhancing, boosting, or stimulating transfection or transduction of a mammalian cell with foreign DNA, comprising contacting the cell with an effective amount of the foreign DNA, under conditions suitable for transfection or transduction to occur, in the presence of an effective amount or concentration of any of:
(a) a compound of formula (IA) or (IB)
(e) a compound of formula (V)
(f) a compound of formula (VI)
(g) a compound of formula (VII)
(h) any of the following compounds: [chemical structure] [chemical structure]
or any salt thereof.

20. (canceled)

21. The method of claim 19 wherein the foreign DNA comprises a single-stranded or double-stranded DNA fragment, a plasmid, a cosmid, a synthetic chromosome, or engineered viral DNA.

22. The method of claim 19 wherein the foreign DNA is contacted with the target cell in the presence of the compound and further in the presence of a transfection adjuvant, or with electroporation, or with nucleofection, or any combination thereof.

23. The method of claim 22 wherein the transfection adjuvant comprises a cationic lipid, a cationic polymer, a cationic peptide, a pegylated liposome, or a combination thereof.

24. The method of claim 16 further comprising inhibiting the degradation of foreign DNA within a eukaryotic cell, comprising contacting the cell comprising the foreign DNA, under conditions suitable for transfection or transduction to occur, with an effective amount or concentration of a compound of formula (IA), (IB), (V), (VI), or (VII).

25. A The method of claim 16 further comprising treating a genetic disease in a patient afflicted therewith, the method comprising contacting a cell or tissue, in vivo in the body of a patient afflicted with the genetic disease, with a curative foreign DNA, under conditions suitable for transfection or transduction to occur, in the presence of an effective amount of any of the transfection-enhancing compounds of formula (IA), (IB), (V), (VI), or (VII).

26. (canceled)

27. (canceled)

28. The method of claim 19 wherein the foreign DNA comprises a single-stranded or double-stranded DNA fragment, a plasmid, a cosmid, a synthetic chromosome, or engineered viral DNA, wherein the foreign DNA incorporates an engineered DNA sequence or sequences.

29. The method of claim 25 wherein the curative foreign DNA comprises plasmid DNA, wherein the plasmid DNA incorporates an engineered DNA sequence or sequences wherein the engineered sequence or sequences is/are adapted to code for correction of a genetic deficiency of the patient.

30. A kit comprising a compound of claim 19, suitably packaged, and, optionally, instructional material, further optionally comprising foreign DNA and a transfection adjuvant, for transfection of a target cell.

31.-32. (canceled)
